(19) United States
(12) Patent Application Publication
(10) Pub. No.: US 2025/0241594 A1
(43) Pub. Date: Jul. 31, 2025

(54) BIOLOGICAL SIGNAL DETECTION MODULE AND BIOLOGICAL SIGNAL DETECTION SYSTEM

(71) Applicants: DENSO CORPORATION, Kariya-city (JP); TOYOTA JIDOSHA KABUSHIKI KAISHA, Toyota-shi (JP); MIRISE Technologies Corporation, Nisshin-shi (JP)

(72) Inventors: Yuki ANNO, Nisshin-shi (JP); Tomoki TANEMURA, Nisshin-shi (JP); Ippei FUKUTOMI, Machida-shi (JP)

(21) Appl. No.: 19/015,921

(22) Filed: Jan. 10, 2025

(30) Foreign Application Priority Data
Jan. 26, 2024 (JP) 2024-009859

(51) Int. Cl. A61B 5/00; A61B 5/0205; A61B 5/11; A61B 7/04

(52) U.S. Cl. CPC A61B 5/702; A61B 5/0205; A61B 5/7203; A61B 5/1101; A61B 7/04; A61B 2560/0443; A61B 2562/066

(57) ABSTRACT

A biological signal detection module includes: a plurality of sensors that detects a biological signal of a subject; and a support on which the plurality of sensors are disposed. The support is to be placed at a position with which a chest or a back of the subject comes into contact in an article that comes into contact with an upper body of the subject. Each of the plurality of sensors has a detection range of the biological signal that is smaller than an organ including a source of the biological signal.

### CROSS REFERENCE TO RELATED APPLICATION

The present application claims the benefit of priority from Japanese Patent Application No. 2024-009859 filed on Jan. 26, 2024. The entire disclosures of the above application are incorporated herein by reference.

### TECHNICAL FIELD

The present disclosure relates to a biological signal detection module and a biological signal detection system.

### BACKGROUND

It has been known a technique for detecting biological signals without requiring a subject to wear a sensor and without restraining the subject. For example, it has been known a technique for detecting biological signals using multiple sensors arranged across a living body.

### SUMMARY

The present disclosure provides a biological signal detection module and a biological signal detection system. According to an aspect, the biological signal detection module includes a plurality of sensors that detects a biological signal of a subject and a support on which the plurality of sensors are disposed. The support is to be placed at a position with which a chest or a back of the subject comes into contact in an article that comes into contact with an upper body of the subject. Each of the plurality of sensors has a detection range of the biological signal that is smaller than an organ including a source of the biological signal.

### BRIEF DESCRIPTION OF THE DRAWINGS

Objects, features and advantages of the present disclosure will become more apparent from the following detailed description made with reference to the accompanying drawings, in which like parts are designated by like reference numbers and in which:

FIG. 1 is a schematic diagram showing a configuration of a biological signal detection system according to a first embodiment;

FIG. 2 is an explanatory diagram showing an example of an arrangement of a plurality of sensors;

FIG. 3 is a schematic diagram showing a configuration of a biological signal acquisition device;

FIG. 4 is an explanatory diagram showing an example of an arrangement of a plurality of sensors of a biological signal detection system according to a second embodiment;

FIG. 5 is a schematic diagram showing a configuration of a biological signal detection system according to a third embodiment;

FIG. 6 is an explanatory diagram showing a biological signal detection module according to a fourth embodiment;

FIG. 7 is an explanatory diagram showing a biological signal detection module according to a fifth embodiment; and

FIG. 8 is an explanatory diagram of a biological signal detection module according to another embodiment.

### DETAILED DESCRIPTION

For example, there is a technique for detecting biological signals without requiring a subject to wear a sensor and without restraining the subject. Also, there is a technique for detecting biological signals using multiple sensors arranged across a living body.

When the area where one sensor contacts a living body is large, the signal-to-noise (S/N) ratio of the signal to be desired in output signals is likely to deteriorate. Therefore, there is a demand for a technique that can measure the biological signal in a small contact area near the source of the biological signal.

According to an aspect of the present disclosure, a biological signal detection module includes: a plurality of sensors that detects a biological signal of a subject; and a support on which the plurality of sensors are disposed. The support is to be placed at a position with which a chest or a back of the subject comes into contact, in an article that comes into contact with an upper body of the subject. Each of the plurality of sensors has a detection area of the biological signal that is smaller than an organ containing a source of the biological signal.

In the biological signal detection module according to the aspect, the biological signal can be measured in a narrow contact range near the source of the biological signal.

Hereinafter, embodiments of the present disclosure will be described with reference to the drawings.

### First Embodiment

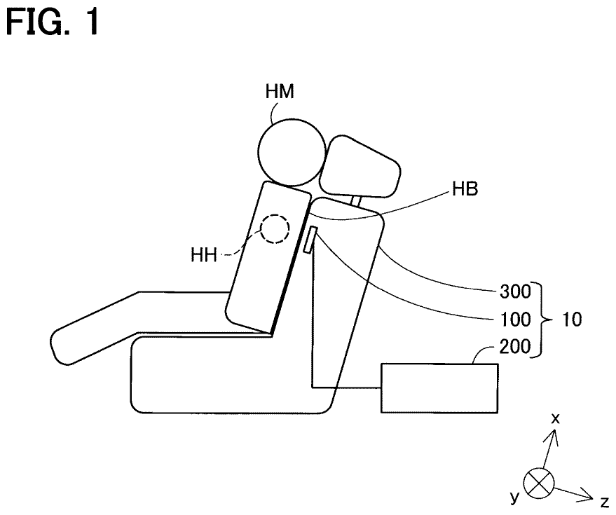

A biological signal detection system 10 shown in FIG. 1 detects a biological signal of a subject HM, i.e., a person to be measured. The biological signal is a signal that indicates a vibration generated from the subject HM. In the present embodiment, the biological signal indicates a cardiac sound of a heart HH of the subject HM, which includes intermittent mechanical or acoustic vibrations in the heart HH, or a cardiac motion, which includes vibrations caused by the force of blood pumped by the heart HH. The mechanical or acoustic vibrations in the heart HH are produced by the opening and closing motions of valves of the heart HH, or the movement of blood into or out of the heart HH. The cardiac sound is the sound generated in association with the beating of the heart HH, and is an elastic wave that propagates through a medium.

Under normal circumstances, the cardiac sound is a periodic signal consisting of a first cardiac sound generated by the closure of the left and right atrioventricular valves at the beginning of ventricular systole, and a second cardiac sound generated by the closure of the aortic and pulmonary valves immediately after the ventricular systole. Typically, the first cardiac sound is low and long, and the second cardiac sound is high and short. The period from the first cardiac sound to the second cardiac sound is the systole of the heart HH, and the period from the second cardiac sound to the first cardiac sound of the next cycle is the diastole of the heart HH. The period from the first cardiac sound to the next first cardiac sound or the period from the second cardiac sound to the next second cardiac sound corresponds to the heart rate interval (HRI), and the number of first cardiac sounds or the number of second cardiac sounds per minute corresponds to the heart rate.

The biological signal detection system 10 includes a biological signal detection module 100, a biological signal acquisition device 200, and a seat 300. The biological signal detection system 10 does not care whether the subject HM is wearing clothing when acquiring the biological signal. FIG. 1 shows an x-axis, a y-axis, and a z-axis which are perpendicular to each other. The x-axis corresponds to a height direction of the subject HM seated on the seat 300, and also corresponds to a height direction of a support 120 described below. The y-axis corresponds to a width direction of the subject HM seated on the seat 300, and also corresponds to a width direction of the support 120. The z-axis corresponds to a thickness direction of the subject HM seated on the seat 300, and also corresponds to a thickness direction of the support 120. These axes correspond to the axes shown in FIG. 1 and the subsequent figures.

The biological signal detection module 100 is placed on the seat 300. The biological signal detection module 100 acquires the biological signal from a back HB of the subject HM who is seated on the seat 300. In the present embodiment, the biological signal detection module 100 is embedded in a recess provided in the seat 300 and is fixed therein by friction with the material constituting the seat 300. Note that it is not always necessary that the biological signal detection module 100 is embedded inside the seat 300. The biological signal detection module 100 may be fixed to the front or back surface of the seat 300.

The biological signal detection module 100 may be manufactured integrally with the seat 300, or may be retrofitted to a ready-made article. In the present disclosure, “retrofit” means that it is not attached to or incorporated into the article at the time of manufacture or installation of the article, but is attached or placed on the article independently of the manufacture or use of the article.

In the present embodiment, for example, the seat 300 is a driver's seat, and the subject HM is a driver seated on the seat 300. The seat 300 is an example of an article that comes into contact with the upper body of the subject HM. Examples of the “article that comes into contact with the upper body of the subject” include various articles used when a person lies down, such as bedding, beds, futons, sheets, examination tables, hospital beds, mats, mattresses, floors, sheets, bed pads, and sofa beds, and various articles used when a person sits on, such as chairs, floor chairs, and sofas. Examples of the “article that comes into contact with the upper body of the subject” include furniture, medical equipment, and the like, and also include any item that can be used when a person is lying down or sitting, regardless of its purpose. In addition, the “article that comes into contact with the upper body of the subject” is not limited to the entire article, but also be a part of the article, such as the back of a chair, a part of a backrest, or a part of a mattress.

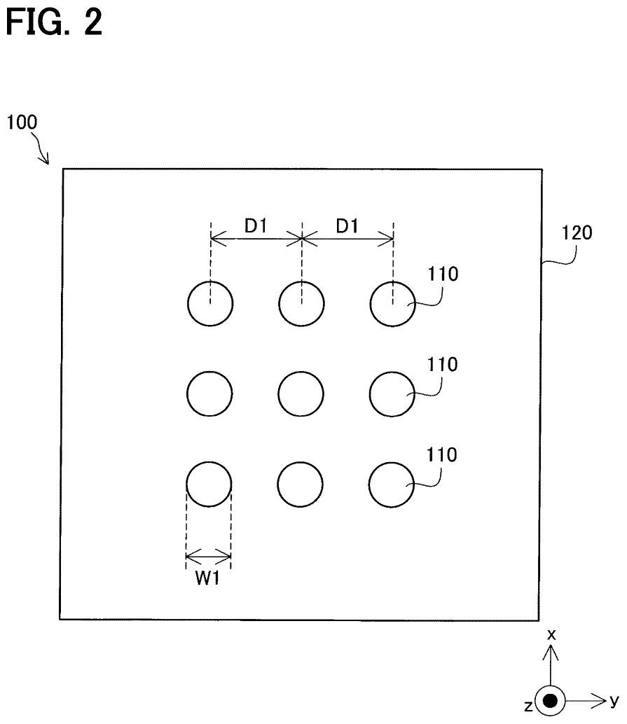

As shown in FIG. 2, the biological signal detection module 100 of the present embodiment includes a plurality of sensors 110 and a support 120. FIG. 2 is a plan view of the biological signal detection module 100 when viewed along the thickness direction (z-axis direction) of the support 120.

The sensor 110 detects the biological signal of the subject HM. In the present embodiment, the sensor 110 is a piezoelectric sensor capable of outputting a vibration waveform including vibrations as the biological signal. The width W1 of the sensor 110 is 4 cm. In the present embodiment, the width W1 is the length (dimension) of the sensor 110 in the width direction of the support 120. The width W1 is the diameter of the sensor 110. From the viewpoint of accurately detecting vibrations corresponding to the biological signal, it is preferable that the sensor 110 has a high sensitivity so as to be able to detect minute signals, such as voltages in the μV range. In the present embodiment, the frequency band of the biological signal detected by the sensor 110 is 10 Hz or more and 100 Hz or less.

The detection range of the sensor 110 is smaller than an organ that includes the source of the biological signal. The organ including the source of the biological signal is an organ having a valve, tissue, or blood vessel, which is the source of the biological signal. An example of the organ including the source of the cardiac sound or cardiac motion is the heart HH, and an example of the organ including the source of vibration associated with breathing is the lung. In the present embodiment, the detection range of the sensor 110 is smaller than the heart HH.

The biological signal detected by the sensor 110 is provided to an analog-to-digital (AD) converter (not shown) as an analog voltage signal of the vibration waveform, converted into a digital signal, and output to the biological signal acquisition device 200 via wired or wireless communication. The wireless communication can be realized, for example, by a wireless connection through a wireless local network (LAN) conforming to the IEEE 802.11 standard or wireless communication using Bluetooth (registered trademark).

The support 120 is a member on which the multiple sensors 110 are arranged. The support 120 is placed at a position on the seat 300 with which the chest or back HB of the subject HM comes into contact. In the present embodiment, the support 120 is a sheet-like member having a thickness of 1 mm. The support 120 is formed using, for example, silicone rubber, from the viewpoint of suppressing any discomfort felt by the subject HM when he/she is seated on the seat 300. When a thin silicone rubber is used as the support 120, it can be deformed to fit the shape of the body of the subject HM, thereby reducing any discomfort felt by the subject HM.

In the present embodiment, the multiple sensors 110 are arranged on the support 120 at an equal interval and in a 3×3 grid pattern in the height direction (x-axis direction) and width direction (y-axis direction) of the support 120. The distance D1 between adjacent sensors 110 is 10 cm. It is not always necessary that the distance D1 is precisely even between the sensors 110. The sensors 110 may be arranged to be substantially evenly spaced, due to manufacturing or other inconsistencies. The sensors 110 may be substantially evenly spaced including an error range of about +/−5%. It is preferable that the multiple sensors 110 are arranged at multiple locations opposing to the organ including the source of the biological signal in the biological signal detection module 100. The length and width of a typical adult lung are about 24 cm and 10 cm. Additionally, the length and width of a typical adult heart are approximately 10 cm. Therefore, the distance D1 between adjacent sensors 110 is preferably 24 cm or less, which is less than the length of the organ including the source of the biological signal, and more preferably 10 cm or less, which is less than the width of the organ including the source of the biological signal. In such a case, it is possible to increase the probability that each of the multiple sensors 110 can acquire the biological signal. In the present embodiment, the length of the heart HH, as the organ including the source of biological signals, corresponds to the size of the support 120 in the longitudinal direction. Moreover, the width of the heart HH corresponds to the size of the support 120 in the width direction.

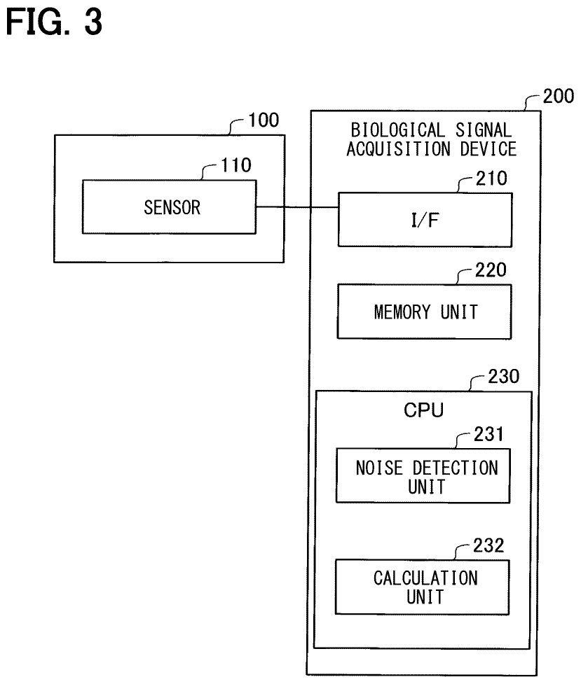

The biological signal acquisition device 200 shown in FIG. 3 is a device that acquires the biological signals detected by the sensors 110. The biological signal acquisition device 200 is configured by a computer including an input/output interface 210, a memory unit 220 made of a ROM and a RAM, and a CPU 230. The input/output interface 210, the memory unit 220, and the CPU 230 are connected to each other so as to enable bidirectional communications therebetween.

The input/output interface 210 is connected to the biological signal detection module 100. The input/output interface 210 receives the biological signals from the multiple sensors 110 included in the biological signal detection module 100.

The CPU 230 executes a program pre-installed in the memory unit 220 to realize the functions as a noise detection unit 231 and a calculation unit 232. However, some or all of the functions of these units may be realized by hardware circuits.

The noise detection unit 231 detects noise in the biological signal. When the biological signal includes the cardiac sound or the cardiac motion, the noise includes vibrations generated from a living body, such as breathing, pulse, organ movement, fetal movements, and bodily movements (body movements), other than the cardiac sound and the cardiac motion which are the biological signals. The noise detection unit 231 detects, for example, an amplitude equal to or less than a predetermined threshold as the noise. The noise detection unit 231 may use a learning model generated by machine learning.

The calculation unit 232 calculates the period of the biological signal. The period of the biological signal is the period from one peak to the next peak, when the biological signal is shown on a graph with the amplitude obtained by wavelet transforming the biological signal on the vertical axis and time on the horizontal axis. The period of the biological signal is, for example, the period from the first cardiac sound to the next first cardiac sound or the period from the second cardiac sound to the next second cardiac sound. In the present embodiment, the calculation unit 232 calculates the period of the biological signal by using the biological signal having the largest intensity among the biological signals detected by the multiple sensors 110.

According to the first embodiment of the biological signal detection system 10 described above, the detection range of the biological signal of the sensor 110 disposed on the support 120, which is placed at a position in contact with the back HB of the subject HM, is smaller than the heart HH, which includes the source of the biological signal. Therefore, the biological signal can be measured in a narrow contact area near the source of the biological signals. Accordingly, it is possible to suppress the deterioration of the S/N ratio.

The multiple sensors 110, each having the width W1 of 4 cm, are arranged in a grid pattern, and the distance D1 between adjacent sensors 110 is 10 cm. The distance D1 is, for example, a distance between the centers of the adjacent sensors 110. More specifically, nine sensors 110 are arranged within an area of 24 cm square. The heart HH, which has the size of approximately 10 cm square and includes the source of the biological signal, is located within the 24 cm square area in which the sensors 110 are arranged. Therefore, there is a high probability that the sensor(s) 110 are located near the heart HH, and the biological signals can be measured near the heart HH.

Further, the calculation unit 232 calculates the period of the biological signal by using the biological signal having the highest intensity or the biological signal with the highest ratio to noise among the biological signals detected by the multiple sensors 110. In this case, it is possible to reduce the processing load in calculating the period of the biological signal.

### Second Embodiment

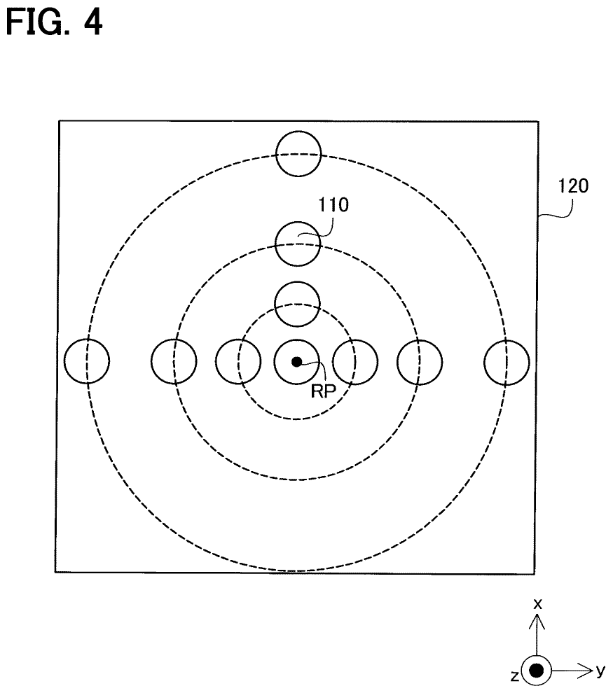

A biological signal detection module according to a second embodiment shown in FIG. 4 is different from the biological signal detection module 100 of the first embodiment in that the sensors 110 are not arranged at an equal interval on the support 120. Since the configuration of the biological signal detection system 10 of the second embodiment is the same as the configuration of the biological signal detection system 10 of the first embodiment, a description of the configuration of the biological signal detection system 10 will be omitted.

As shown in FIG. 4, the multiple sensors 110 are arranged more sparsely as they are away from a predetermined point RP, that is, as the function of distance from the predetermined point RP. The multiple sensors 110 are arranged in a fan shape. The predetermined point RP is preferably located at a position that faces the center of the heart HH in the z-axis direction.

The positional relationship between the sensor 110 and the heart HH varies depending on the physique of the subject HM. According to the biological signal detection system 10 of the second embodiment described above, the multiple sensors 110 are arranged more sparsely as they are away from the predetermined point RP. Therefore, if the subject HM has a small physique, the biological signals can be detected by the sensors 110 near the predetermined point RP. If the physique of the heart HH is large, the biological signals can be detected also by the sensors 110 located away from the predetermined point RP. Therefore, the biological signals can be detected without changing the installation position of the biological signal detection module 100 according to the subject HM.

### Third Embodiment

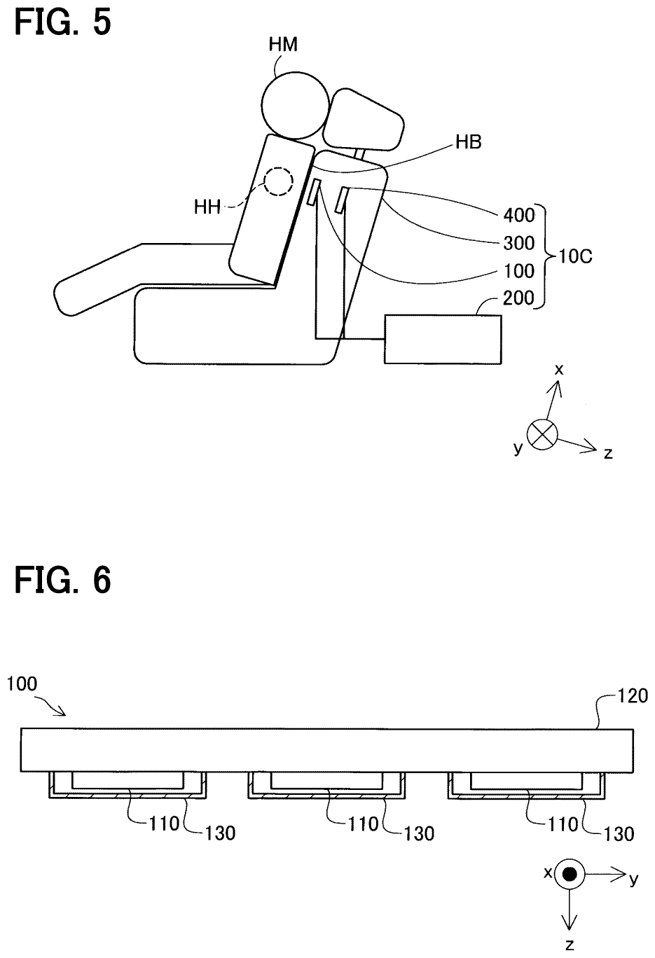

A biological signal detection system 10C according to a third embodiment shown in FIG. 5 is different from the biological signal detection system 10 of the first embodiment in that it includes a noise sensor 400, but the other configurations are the same.

The noise sensor 400 is a sensor that detects a signal indicative of vibration. In the present embodiment, the noise sensor 400 is a piezoelectric sensor similar to the sensor 110. The noise sensor 400 is arranged on the seat 300, on the side opposite to a contact side of the seat 300 with which the subject HM comes into contact with respect to the support 120 in the thickness direction (z-axis direction) of the support 120. In other words, the noise sensor 400 is arranged behind the support 120. The noise sensor 400 is arranged at a position facing the sensor 110 in the thickness direction of the support 120. Therefore, the amplitude of the biological signal detected by the noise sensor 400 is likely to be smaller and at a later timing than the amplitude of the biological signal detected by the sensor 110.

In the present embodiment, when the timing of occurrence of a first amplitude of the biological signal detected by the multiple sensors 110 is later than the timing of occurrence of a second amplitude of the signal detected by the noise sensor 400 corresponding to the first amplitude, or when the intensity of the first amplitude is smaller than the intensity of the second amplitude, the noise detection unit 231 detects the first amplitude as noise. The first amplitude can be determined arbitrarily by the noise detection unit 231.

According to the biological signal detection system 10C of the third embodiment described above, the noise detection unit 231 detects the noise by using the signal detected by the noise sensor 400. Therefore, the noise detection unit 231 can detect the noise with high accuracy when the source of the noise is located on the side opposite to the contact portion of the seat 300 with which the subject HM comes into contact with respect to the support 120 in the thickness direction.

### Fourth Embodiment

A biological signal detection system 10 according to a fourth embodiment shown in FIG. 6 is different from the biological signal detection system 10 of the first embodiment in that the biological signal detection module 100 includes a plurality of covers 130, but the other configurations are the same. In the present embodiment, the biological signal detection module 100 has nine covers 130.

The cover 130 is a member that covers the portion of the sensor 110 that is not in contact with the support 120 without contacting the sensor 110. The covers 130 are correspondingly provided for the sensors 110. Each cover 130 covers the corresponding sensor 110. The cover 130 has a substantially circular shape. The cover 130 preferably has a shape similar to that of the sensor 110. In the present embodiment, the cover 130 is a member made of metal. The cover 130 may be made of resin or plastic.

The resonant frequency of the portion of the support 120 covered by the cover 130 is determined by the weight and the size of the inner diameter of the cover 130. For example, the spring constant of the portion of the support 120 covered by the cover 130 is determined by the size of the inner diameter of the cover 130, and thus the resonant frequency is determined. The weight and the inner diameter of the cover 130 can be determined arbitrarily according to the desired resonant frequency. It is possible to determine the desired resonant frequency so as to amplify the vibration frequency that is desired to be acquired by the sensor 110. The resonant frequency may be determined so as to amplify a frequency that is highly likely to be a biological signal, or so as to amplify a frequency that is less likely to be a biological signal.

According to the biological signal detection system 10 of the fourth embodiment described above, the sensor 110 is covered by the cover 130, and therefore the sensor 110 can be protected. Furthermore, the resonant frequency of the portion of the support 120 covered by the cover 130 can be controlled by adjusting the weight of the cover 130 and the inner diameter of the cover 130.

### Fifth Embodiment

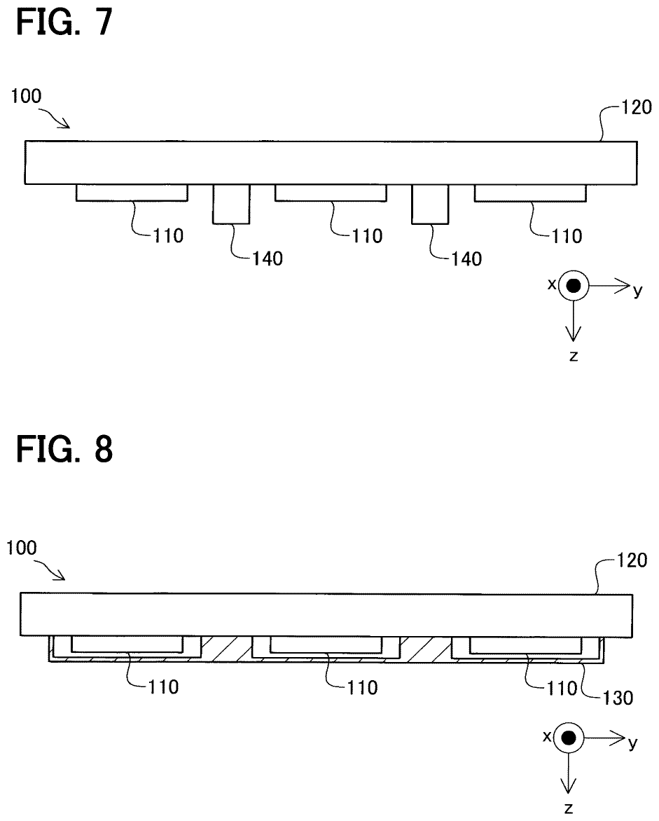

A biological signal detection system 10 according to a fifth embodiment shown in FIG. 7 is different from the biological signal detection system 10 of the first embodiment in that the biological signal detection module 100 includes a weight 140, but the other configurations are the same.

In the present embodiment, the biological signal detection module 100 includes a plurality of weights 140. The weight 140 is attached to the support 120 without contacting the sensor 110. In the present embodiment, the weight 140 is made of metal and is arranged between the sensors 110. The mass of the weight 140 can be determined arbitrarily according to the desired resonant frequency.

According to the biological signal detection system 10 of the fifth embodiment described above, since the support 120 is provided with the weights 140, the resonant frequency of the support 120 including the weights 140 can be controlled by the mass of the weights 140.

### OTHER EMBODIMENTS

(1) In the embodiments described above, the biological signal is the signal indicative of the cardiac sound or cardiac motion. The biological signal is not limited to such examples, and may indicate any vibration generated from a living body, such as respiration or pulse.

(2) In the embodiments described above, the sensor 110 is the piezoelectric sensor having the width W1 of 4 cm. However, the sensor 110 may be a piezoelectric sensor having any width. The smaller the width of the biological signal detection module 100 is, the lower the probability of acquiring noise is and the more the deterioration of the S/N ratio is suppressed. However, since the detection range of the biological signal detection module 100 is reduced, it will be difficult to detect the biological signal. Therefore, it is preferable to appropriately select the size depending on the size of the organ including the source of the biological signal and the size of the biological signal to be acquired. Furthermore, the sensor 110 is not limited to a piezoelectric sensor. Various sensors capable of detecting vibrations, such as an accelerometer, a diaphragm, or a microphone, can be used as the sensor 110.

(3) In the embodiments described above, the sensor 110 is arranged on the article that comes into contact with the chest or back HB of the subject HM. However, it is not always necessary that the sensor 110 is arranged on the article that comes into contact with the chest or back HB of the subject HM.

(4) In the embodiments described above, the multiple sensors 110 are arranged in the grid pattern. However, the multiple sensors 110 may be arranged in any desired manner. The multiple sensors 110 can be arranged, for example, in a concentric pattern. For example, the multiple sensors 110 are arranged such that a direction connecting the centers of the detection ranges of any two of the multiple sensors 110 intersects with the height direction (x-axis direction) of the support 120. This increases the likelihood that the sensors 110 will be positioned closer to the heart HH than the case where the multiple sensors 110 are only positioned longitudinally linearly along the subject HM. As another example, the multiple sensors 110 are arranged such that a direction connecting the centers of the detection ranges of any two of the multiple sensors 110 intersects with the width direction (y-axis direction) of the support 120. This increases the likelihood that the sensors 110 will be positioned closer to the heart HH than the case where the multiple sensors 110 are only positioned transversely linearly across the subject HM.

(5) In the embodiments described above, the frequency band of the biological signal detected by the sensor 110 is 10 Hz or more and 100 Hz or less. However, the sensor 110 may detect a biological signal in any band. When the biological signal is respiration, the band detected by the sensor 110 is preferably 0.2 Hz or more and 0.5 Hz or less.

(6) In the embodiments described above, the support 120 is a silicone sheet-like member having a thickness of 1 mm. However, the support 120 is not limited to this and may be a sheet-like member of any thickness. The support 120 preferably has a thickness of 0.5 mm or more and 20 mm or less. Further, the support 120 may be, for example, a depressed resin member that forms an internal space for housing the sensor 110 therein. In this case, the support 120 houses the sensor 110 and a catalyst other than a gas, such as a liquid, solid, or gel.

(7) In the embodiments described above, the support 120 may be formed with air vent holes at positions where the sensors 110 are not provided. This can suppress the biological signal detection module 100 from deteriorating due to sweat, stuffiness, and the like. Further, even when the seat 300 is configured to blow air to the subject HM seated on the seat 300, it is possible to avoid the air being blocked by the support 120.

(8) In the embodiments described above, the calculation unit 232 calculates the period of the biological signal by using the biological signal with the highest intensity among the biological signals detected by the multiple sensors 110. Without being limited thereto, the calculation unit 232 may calculate the period of the biological signal by using, for example, the biological signal having the highest ratio to noise among the biological signals detected by the multiple sensors 110. The calculation unit 232 may also calculate the period of the biological signal by averaging the periods of the biological signals detected by the multiple sensors 110.

(9) In the first, second and fourth embodiments described above, the biological signal detection system 10 may not include the noise detection unit 231. In this case, the calculation unit 232 calculates the period from the biological signal using a filter that is generated in advance and that attenuates noise, for example.

(10) In the third embodiment described above, the noise sensor 400 is arranged at a position facing the sensor 110 in the thickness direction (z-axis direction) of the support 120. Without being limited thereto, the noise sensor 400 may be arranged at a position not facing the sensor 110 in the thickness direction of the support 120. Furthermore, a plurality of noise sensors 400 may be provided in the planar direction of the support 120, or a plurality of noise sensors 400 may be provided in the thickness direction of the support 120.

(11) In the fourth embodiment described above, the biological signal detection module 100 has the covers 130 provided correspondingly for the sensors 110. Without being limited thereto, for example, as shown in FIG. 8, the cover 130 may be provided so as to cover all of the sensors 110 provided in the biological signal detection module 100. Since the area of the cover 130 is increased, the pressure applied to the cover 130 can be dispersed.

(12) In the fifth embodiment described above, the weight 140 is provided between the sensors 110. Without being limited to thereto, the weight 140 may be provided at any position on the support 120. For example, the weight 140 may be provided on the outer periphery of the sensors 110.

The present disclosure should not be limited to the embodiments described above, and various other embodiments may be implemented without departing from the scope of the present disclosure. For example, the technical features in each embodiment corresponding to the technical features in the form described in the summary may be used to solve some or all of the above-described issues, or to provide one of the above-described effects. In order to achieve a part or all, replacement or combination can be appropriately performed. Also, if the technical features are not described as essential in the present specification, they can be deleted as appropriate.

The biological signal acquisition device 200 and the methods thereof described in the present disclosure may be realized by a special purpose computer provided by configuring a processor and memory programmed to perform one or more functions embodied in a computer program. Alternatively, the noise detection unit 231 and the calculation unit 232 and the methods thereof described in the present disclosure may be implemented by a special purpose computer provided by configuring a processor with one or more dedicated hardware logic circuits. Alternatively, the noise detection unit 231 and the calculation unit 232 and the methods thereof described in the present disclosure may be implemented by one or more dedicated computers configured by a combination of a processor and memory programmed to perform one or more functions and a processor configured by one or more hardware logic circuits. The computer program may be stored in a computer-readable non-transitory tangible recording medium as an instruction executed by the computer.

What is claimed is:

1. A biological signal detection module comprising:
a plurality of sensors that detects a biological signal of a subject; and
a support on which the plurality of sensors are disposed, the support being to be placed at a position with which a chest or a back of the subject comes into contact in an article that comes into contact with an upper body of the subject, wherein
each of the plurality of sensors has a detection range of the biological signal that is smaller than an organ including a source of the biological signal.

2. The biological signal detection module according to claim 1, wherein
the plurality of sensors is disposed so that a direction connecting the centers of the detection ranges of any two of the plurality of sensors intersects with a height direction of the support.

3. The biological signal detection module according to claim 1, wherein
the plurality of sensors is disposed so that a direction connecting the centers of the detection ranges of any two of the plurality of sensors intersects with a width direction of the support.

4. The biological signal detection module according to claim 2, wherein
the plurality of sensors is disposed so that a distance between adjacent sensors among the plurality of sensors is equal to or less than a length of the organ.

5. The biological signal detection module according to claim 4, wherein
the distance is equal to or less than a width of the organ.

6. The biological signal detection module according to claim 5, wherein
the plurality of sensors includes a piezoelectric sensor having a width of 4 cm or less.

7. The biological signal detection module according to claim 2, wherein
the plurality of sensors is arranged at an equal interval.

8. The biological signal detection module according to claim 2, wherein
the plurality of sensors is arranged so that a distance between adjacent sensors increase as a function of distance from a predetermined point of the support.

9. The biological signal detection module according to claim 6, wherein
the support has a thickness of 0.5 mm or more and 20 mm or less.

10. The biological signal detection module according to claim 6, further comprising:
a weight disposed on the support.

11. The biological signal detection module according to claim 6, further comprising:
a cover that covers a portion of at least one of the plurality of sensors without contacting the at least one of the plurality of sensors, the portion being not in contact with the support.

12. A biological signal detection system comprising:
the biological signal detection module according to claim 1;
a noise detection unit that detects noise in the biological signal;
a noise sensor that detects a signal and is disposed in the article at a position opposite to a contact portion of the article with which the subject comes into contact with respect to the support in a thickness direction of the support, wherein
when a timing of occurrence of a first amplitude of the biological signal detected by the plurality of sensors is later than a timing of occurrence of a second amplitude of the signal detected by the noise sensor corresponding to the first amplitude, or when an intensity of the first amplitude is smaller than an intensity of the second amplitude, the noise detection unit detects the first amplitude as the noise.

13. A biological signal detection system comprising:
the biological signal detection module according to claim 1; and
a calculation unit that calculates a period of the biological signal using the biological signal having a largest intensity or a highest ratio to noise among a plurality of the biological signals detected by the plurality of sensors.

14. The biological signal detection module according to claim 1, wherein
the biological signal indicates a cardiac sound or a cardiac motion, and
the biological signal has a frequency band of 10 Hz or more and 100 Hz or less.
